(19) United States
(12) Patent Application Publication
(10) Pub. No.: US 2023/0330138 A1
(43) Pub. Date: Oct. 19, 2023

(54) TREATMENT OF CARTILAGE DEGENERATION USING MYELOID SUPPRESSOR CELLS AND EXOSOMES DERIVED THEREOF

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Phoenix, AZ (US)

(73) Assignee: CREATIVE MEDICAL TECHNOLOGIES, INC., Phoenix, AZ (US)

(21) Appl. No.: 18/300,233

(22) Filed: Apr. 13, 2023

Related U.S. Application Data
(60) Provisional application No. 63/331,186, filed on Apr. 14, 2022.

(51) Int. Cl. A61K 35/15; A61K 35/28; A61K 9/127; A61K 35/545; A61N 5/067

(52) U.S. Cl. CPC A61K 35/15; A61K 35/28; A61K 9/127; A61K 35/545; A61N 5/067

(57) ABSTRACT

Disclosed are therapeutic means of suppressing degeneration of cartilage in conditions such as arthritis or other degenerative pathologies through administration of myeloid derived suppressor cells or exosomes produced by said cells. In one embodiment myeloid derived suppressor cells are generated by intravenous administration of a mobilizing cytokines followed by collection of myeloid derived suppressor cells through an apheresis means, followed by subsequent introduction into the intra-articular space. In other embodiments allogeneic myeloid derived suppressor cells are administered from an allogeneic source such as umbilical cord blood mononuclear cells.

### CROSS-REFERENCE TO RELATED APPLICATIONS

This application claims priority to U.S. Provisional Application Ser. No. 63/331,186, titled “Treatment of Cartilage Degeneration Using Myeloid Suppressor Cells and Exosomes Derived Thereof” filed Apr. 14, 2022, which is hereby incorporated by reference in its entirety.

### FIELD OF THE INVENTION

The teachings herein are directed to methods of enchancing stem cells in order to administer to patients suffering from cartilage degeneration.

### BACKGROUND

The possibility of utilizing immune modulatory cells for the stimulation of regenerative activities in the body is a tantalizing possibility that to our knowledge has not been explored. In pregnancy it is known that the genetic difference between the maternal and paternal genomes is responsible for the size of the placenta with larger genetic differences eliciting more profound placental formation and angiogenesis. To our knowledge, to date, no one has explored the possibility of therapeutically utilizing immunological cells to stimulate regeneration in the context of healing or inducing acceleration of recovery of a pathology.

In the field of immunology and immune modulation, myeloid-derived suppressor cells (MDSC) are a heterogeneous population of immature myeloid cells that are similar to the Natural Suppressor cells discovered by Sharwan Singhal.

Regardless if they are termed myeloid derived suppressor cells or natural suppressors, these cells accumulate to a great extent in cancer patients and play a major role in regulating immune responses in cance.sup.r42. MDSC suppress T cells activation and proliferation as well as function of natural killer (NK) cells.sup.14,15. Ample evidence links these cells with tumor progression and outcome of the disease in cancer patients.sup. The accumulation of relatively immature and pathologically activated myeloid-derived suppressor cells (MDSC) with potent immunosuppressive activity is common in tumors. MDSC have the ability to support tumor progression by promoting tumor cell survival, angiogenesis, invasion of healthy tissue by tumor cells. There is now ample evidence of the association of accumulation of immune suppressive MDSC with negative clinical outcomes in various cancers MDSC have been implicated in resistance to anticancer therapies with kinase inhibitor chemotherapy.

### SUMMARY

Preferred embodiments are directed to methods of treating cartilage degeneration comprising administration of myeloid derived suppressor cells and/or exosomes generated under stimulated or unstimulated conditions from said myeloid derived suppressor cells.

Preferred methods include embodiments wherein myeloid derived suppressor cells express CD90.

Preferred methods include embodiments wherein myeloid derived suppressor cells express Gr1.

Preferred methods include embodiments wherein myeloid derived suppressor cells express CD31.

Preferred methods include embodiments wherein myeloid derived suppressor cells express VEGF-receptor.

Preferred methods include embodiments wherein myeloid derived suppressor cells express interleukin-3 receptor.

Preferred methods include embodiments wherein myeloid derived suppressor cells express PECAM-1.

Preferred methods include embodiments wherein myeloid derived suppressor cells express CD133.

Preferred methods include embodiments wherein myeloid derived suppressor cells express CD34.

Preferred methods include embodiments wherein myeloid derived suppressor cells express LFA-1.

Preferred methods include embodiments wherein myeloid derived suppressor cells are capable of inhibiting T cell proliferation.

Preferred methods include embodiments wherein said T cell proliferation is multiplication of CD4 T cells.

Preferred methods include embodiments wherein said T cell proliferation is multiplication of CD8 T cells.

Preferred methods include embodiments wherein said T cell proliferation is multiplication of CD56 T cells.

Preferred methods include embodiments wherein T cell proliferation is stimulated by interleukin-2.

Preferred methods include embodiments wherein T cell proliferation is stimulated by interleukin-7.

Preferred methods include embodiments wherein T cell proliferation is stimulated by interleukin-15.

Preferred methods include embodiments wherein T cell proliferation is stimulated by interleukin-18.

Preferred methods include embodiments wherein T cell proliferation is stimulated by a lectin.

Preferred methods include embodiments wherein said lectin is phytohemagglutinin.

Preferred methods include embodiments wherein said lectin is concanavalin A.

Preferred methods include embodiments wherein said lectin is pokeweed mitogen.

Preferred methods include embodiments wherein said lectin is GNA.

Preferred methods include embodiments wherein said T cell proliferation is stimulated by anti-CD3 antibody.

Preferred methods include embodiments wherein said T cell proliferation is stimulated by anti-CD28 antibody.

Preferred methods include embodiments wherein said T cell proliferation is stimulated by a combination of anti-CD3 and anti-CD28 antibody.

Preferred methods include embodiments wherein said myeloid derived suppressor cells are progenitors of granulocytes.

Preferred methods include embodiments wherein said myeloid derived suppressor cells are progenitors of monocytes.

Preferred methods include embodiments wherein said myeloid derived suppressor cells are progenitors of monocytes and granulocytes.

Preferred methods include embodiments wherein said myeloid derived suppressor cells are progenitors of dendritic cells.

Preferred methods include embodiments wherein said myeloid derived suppressor cells are collected from apheresed product after mobilization.

Preferred methods include embodiments wherein said mobilization is achieved by administration of G-CSF.

Preferred methods include embodiments wherein said mobilization is achieved by administration of AMD 3100.

Preferred methods include embodiments wherein said mobilization is achieved by administration of CTCE-9908.

Preferred methods include embodiments wherein said mobilization is achieved by administration of FTY720.

Preferred methods include embodiments wherein said mobilization is achieved by administration of Flt3 ligand.

Preferred methods include embodiments wherein said mobilization is achieved by administration of SCF.

Preferred methods include embodiments wherein said mobilization is achieved by administration of S100A9.

Preferred methods include embodiments wherein said mobilization is achieved by administration of GM-CSF.

Preferred methods include embodiments wherein said mobilization is achieved by administration of M-CSF.

Preferred methods include embodiments wherein said exosomes are derived from said cell population is autologous.

Preferred methods include embodiments wherein said cell population is allogeneic.

Preferred methods include embodiments wherein said cell population is xenogenic.

Preferred methods include embodiments wherein said cell population is bone marrow aspirate/mononuclear cells.

Preferred methods include embodiments wherein said cell population is mesenchymal stem cells.

Preferred methods include embodiments wherein said cell population is amniotic stem cells.

Preferred methods include embodiments wherein said cell population is embryonic stem cells.

Preferred methods include embodiments wherein said cell population is inducible pluripotent stem cells.

Preferred methods include embodiments wherein said cell population is a hematopoietic stem cell population.

Preferred methods include embodiments wherein said mesenchymal stem cells express CD90.

Preferred methods include embodiments wherein said mesenchymal stem cells express CD105.

Preferred methods include embodiments wherein said mesenchymal stem cells express c-met.

Preferred methods include embodiments wherein said mesenchymal stem cells express CD133.

Preferred methods include embodiments wherein said mesenchymal stem cells express c-kit.

Preferred methods include embodiments wherein said mesenchymal stem cells express IL-1 receptor.

Preferred methods include embodiments wherein said mesenchymal stem cells express IL-1 receptor antagonist when treated with interferon gamma.

Preferred methods include embodiments wherein said cell population is administered before or after intraarticular laser treatment.

### DETAILED DESCRIPTION OF THE INVENTION

For the purpose of the invention, we disclose that two populations of myeloid derived suppressor cells are capable of stimulating regeneration of injured or degenerated cartilage. The first type of myeloid derived suppressor cells is the monocytic myeloid-derived suppressor cells (M-MDSC) and the second type is polymorphonuclear myeloid-derived suppressor cells (PMN-MDSC). About 20-30% of MDSC consists of monocytic cells, i.e., M-MDSC, and are generally associated with high activity of Arginase-1 and iNOS.sup.10. Two different phenotypes (CD11b.sup.+CD14.sup.-CD15.sup.− and CD33.sup.+ or CD11b.sup.+CD14.sup.+CD33.sup.+ and HLA-DR.sup.lo) are used to characterize these M-MDSC cells depending on the type of cancer. The second population, i.e., PMN-MDSC, are comprised of granulocytic cells and are usually associated with high level of ROS production.sup.36. PMN-MDSC represent the major population of MDSC (about 60-80%) and represent the most abundant population of MDSC in most types of cancer. PMN-MDSC are phenotypically and morphologically similar to neutrophils (PMN) and share the CD11b+ CD14-CD15+/CD66b+ phenotype. The may also be characterized as CD33.sup.+. PMN-MDSC are important regulators of immune responses in cancer and have been directly implicated in promotion of tumor progression. However, the heterogeneity of these cells and lack of distinct markers hampers the progress in understanding of the biology and clinical significance of these cells. One of the major obstacles in the identification of PMN-MDSC is that they share the same phenotype with normal polymorphonuclear cells (PMN).

The administration of myeloid derived suppressor cells may be performed by using the cells themselves or by pre-activating them. In certain embodiments of the invention, small molecules of the invention are used to sustain and enhance the immune suppressive functions of MDSCs by preventing the MDSCs to undergo maturation and terminal differentiation. Through this process the growth factor producing properties of the myeloid suppressor cells are retained and/or enhanced.

For the purpose of the invention, we describe the immature stage of MDSCs as being characterized by low cell surface expression of MHC class II, co-stimulatory molecules, e.g., CD80, CD86, CD40, low CD11c and F4/80. Immature MDSCs arc further characterized by a large nucleus to cytoplasm ratio and an immunosuppressive activity. In some cases enhancement of growth factor properties is produced by treatment of the cells by histone deacetylase inhibitors such as decitabine. In some embodiments of the invention, MDSCs are autologously-derived cells. For example, MDSCs may be isolated from normal adult bone marrow or from sites of normal hematopoiesis, such as the spleen. Obviously, splenic sources of MDSC are difficult in clinical situations. MDSCs are scant in the periphery and are present in a low number in the bone marrow of healthy individuals. However, they are accumulated in the periphery when intense hematopoiesis occurs. Upon distress due to graft-versus-host disease (GVHD), cyclophosphamide injection, or g-irradiation, for example, MDSCs may be found in the adult spleen. Thus, in certain embodiments, MDSCs may be isolated from the adult spleen. MDSCs may also be isolated from the bone marrow and spleens of tumor-bearing or newborn mice. In a preferred embodiment, MDSCs are isolated in vivo by mobilizing MDSCs from hematopoietic stem cells (HSCs) or bone marrow suing stem cell mobilizers such as G-CSF Any suitable stem cell mobilizer or combination of mobilizers is contemplated for use in the present invention. MDSCs may be induced endogenously and/or be collected from the blood e.g., by apheresis, following treatment of a subject or patient with the stem cell mobilizer(s). In certain embodiments, MDSCs can be derived, for example, in vitro from a patient's HSCs, from MHC matching ES cells, induced pluripotent stem (iPS) cells Specifically, isolated hematopoietic stem cells (HSCs) can be stimulated to differentiate into Gr-1+/CD11b+, Gr-1+/CD11b.+/CD115+, Gr-1+/CD11b+/F4/80+, or Gr-1+/CD11b+/CD115+/F4/80+ MDSCs by culturing in the presence of stem-cell factor (SCF) or SCF with tumor factors, which can increase the MDSC population. The culture conditions for mouse and human HSCs are described in detail in U.S. Publication No. 2008/0305079 by Chen. In further embodiments, other cytokines may be used, e.g., VEGF, GM-CSF, M-CSF, SCF, S100A9, TPO, IL-6, IL-1, PGE-2 or G-CSF to stimulate MDSC differentiation from HSCs in vitro. Any one of the cytokines may be used alone or in combination with other cytokines. In still another embodiment, tumor-conditioned media may be used with or without SCF to stimulate HSCs to differentiate into MDSCs. In other embodiments, MDSCs are allogeneic cells, such as MDSCs obtained or isolated from a donor or cell line. MDSC cell lines and exemplary methods for their generation are well known in the art and are described in the literature.

The invention provides administration of myeloid derived suppressor cells, and/or exosomes derived from such cells as a treatment for cartilage degeneration. One of ordinary skill in the art may readily determine the appropriate concentration, or dose of the myeloid derived suppressor cells disclosed herein for therapeutic administration. The ordinary artisan will recognize that a preferred dose is one that produces a therapeutic effect, such as preventing, treating and/or reducing inflammation associated with cartilage diseases, disorders and injuries, in a patient in need thereof. Of course, proper doses of the cells will require empirical determination at time of use based on several variables including but not limited to the severity and type of disease, injury, disorder or condition being treated; patient age, weight, sex, health; other medications and treatments being administered to the patient; and the like. An exemplary dose is in the range of about 0.25-2.0.times.10.sup.6 cells. Other dose ranges include 0.1-10.0.times.10.sup.6,7,8,9,10,11, or 10.sup.12 cells per dose or injection regimen. An effective amount of cells may be administered in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of pharmaceutical composition. Where there is more than one administration of a pharmaceutical composition in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals. A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months. Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days. Cells derived from the methods of the present invention may be formulated for administration according to any of the methods disclosed herein in any conventional manner using one or more physiologically acceptable carriers optionally comprising excipients and auxiliaries. Proper formulation is dependent upon the route of administration chosen. The compositions may also be administered to the individual in one or more physiologically acceptable carriers. Carriers for cells may include, but are not limited to, solutions of normal saline, phosphate buffered saline (PBS), lactated Ringer's solution containing a mixture of salts in physiologic concentrations, or cell culture medium. In further embodiments of the present invention, at least one additional agent may be combined with the cartilage-derived progenitor cells of the present invention for administration to an individual according to any of the methods disclosed herein. Such agents may act synergistically with the cells of the invention to enhance the therapeutic effect. Such agents include, but are not limited to, growth factors, cytokines, chemokines, antibodies, inhibitors, antibiotics, immunosuppressive agents, steroids, anti-fungals, anti-virals or other cell types (i.e. stem cells or stem-like cells, for example AMP cells), extracellular matrix components such as aggrecan, versican hyaluronic acid and other glycosaminoglycans, collagens, etc. Inactive agents include carriers, diluents, stabilizers, gelling agents, delivery vehicles, ECMs (natural and synthetic), scaffolds, and the like. When the cells of the present invention are administered conjointly with other pharmaceutically active agents, even less of the cells may be needed to be therapeutically effective. The timing of administration of myeloid derived suppressor cells 1-based compositions will depend upon the type and severity of the cartilage disease, disorder, or injury being treated. In one embodiment, the cell-based compositions are administered as soon as possible after onset of symptoms, diagnosis or injury. In another embodiment, cell-based compositions are administered more than one time following onset of symptoms, diagnosis or injury. In certain embodiments, where surgery is required, the cell-based compositions are administered at surgery. In still other embodiments, the cell-based compositions are administered at as well as after surgery. Such post-surgical administration may take the form of a single administration or multiple administrations.

In some embodiments, the myeloid derived suppressor cells are administered parenterally to the individual. The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral, intraosseous, intracartilagenous, and intrasternal injection or infusion. Support matrices, scaffolds, membranes and the like into which the cell-based compositions can be incorporated or embedded include matrices which are recipient-compatible and which degrade into products which are not harmful to the recipient. Detailed information on suitable support matrices, etc. can be found in U.S. Pat. Nos. 8,058,066 and 8,088,732, both of which are incorporated herein by reference.

n certain aspects of the invention, the small compound glatiramer acetate (GA) (Copolymer 1/Copaxone) is used to modify MDSC function. In another aspect, a small compound MAP kinase inhibitor is used to modify MDSC function. In yet another aspect, GA and a small compound MAP kinase inhibitor, such as, e.g., a c-Jun N-terminal kinase (JNK) small compound inhibitor, have a surprising synergistic effect on the modulation of MDSC function for the treatment or prevention of alloimmune response and pro-inflammatory immune responses.

In some embodiments MDSC are pre-activated before their administration into tissue possessing degenerated cartilage. We describe the use of small molecules to regulate biological signals in order to alter the properties of MDSC. Signal regulation by small compounds (e.g., small molecule inhibitors) can control cell differentiation and function in a controllable and reproducible manner according to the current invention.

The term “small compound” as used herein refers to compounds, chemicals, small molecules, small molecule inhibitors, or other factors that are useful for modulating MDSC function. Small molecule inhibitors have been used as immunosuppressive and anti-inflammatory drugs. GA (Copolymer 1/Copaxone) is an FDA approved drug for the treatment of multiple sclerosis, a T cell-mediated autoimmune disease. SP600125 is a small compound inhibitor of JNK, which is a downstream molecule of a number of signaling pathways that regulate both innate and adaptive immunity. The present invention is related to the discovery that these small compounds can regulate the suppressive functions of MDSCs to facilitate the establishment of immune tolerance. In one embodiment of the invention GA is administered systemically as a treatment of cartilage degeneration. In another embodiment treatment of osteoarthritis by GA is disclosed. It has been known for a while that GA alone has not been effective for treating autoimmune diseases. Specifically, GA is known to be only partially effective for treating the autoimmune disease multiple sclerosis [Johnson et al. (1995) Neurology 45: 1268-1276]. Moreover, clinical studies using GA for the treatment of IBD were discontinued, because GA failed to treat IBD. The present invention is based on the discovery that administration of GA in combination with MDSCs, or with MDSCs and a MAP kinase inhibitor (e.g., SP600125), is surprisingly effective for the treatment of the autoimmune disease, IBD. It is presently discovered that GA and SP600125 have a synergistic effect in combination. In order to increase therapeutic efficacy in some cases, GA is administered intra-articularly and/or by depot or drug delivery mechanisms in order to enhance the concentration locally without inducing systemic effects. In other embodiments MDSC are first-pretreated before administration of cells intra-articularly. In some embodiments GA is administered together with autologous bone marrow cells.

For practical implementation of the invention, in some embodiments autologous non-expanded cells are provided to a patient with cartilage degeneration while the patient is concurrently receiving the treatment COPAXONE™ which is the brand name for GA (formerly known as copolymer-1). GA, the active ingredient of COPAXONE™, is a random polymer consisting of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively [CAS number 147245-92-9]. The average molecular weight of GA is 4,700 11,000 daltons. Chemically, glatiramer acetate is designated L-glutamic acid polymer with L-alanine, L-lysine and L-tyrosine, acetate (salt). GA is a random polymer composed of tyrosine, glutamic acid, alanine and lysine, that has been used for the treatment of multiple sclerosis, a T cell-mediated autoimmune disease. GA may be obtained from Teva Pharmaceutical Industries (Petach Tikva, Israel). For the practice of the present invention, variants, modified forms and/or derivatives of GA are also contemplated for use in the present invention. One of skill in the art can readily substitute structurally-related amino acids for GA without deviating from the spirit of the invention. The present invention includes polypeptides and peptides which contain amino acids that are structurally related to tyrosine, glutamic acid, alanine or lysine and possess the ability to stimulate polyclonal antibody production upon introduction. Such substitutions retain substantially equivalent biological activity in their ability to suppress autoimmune diseases such as IBD, and alloimmune responses, such as GVHD and organ transplantation rejection. These substitutions are structurally-related amino acid substitutions, including those amino acids which have about the same charge, hydrophobicity and size as tyrosine, glutamic acid, alanine or lysine. For example lysine is structurally-related to arginine and histidine; glutamic acid is structurally-related to aspartic acid; tyrosine is structurally-related to serine, threonine, phenylalanine and tryptophan; and alanine is structurally-related to valine, leucine and isoleucine. These and other conservative substitutions, such as structurally-related synthetic amino acids, are contemplated by the present invention. Any one or more of the amino acids in GA may be substituted with 1- or d-amino acids. As is known by one of skill in the art, 1-amino acids occur in most natural proteins. However, d-amino acids are commercially available and can be substituted for some or all of the amino acids used to make GA. Thus, in some embodiments, the present invention contemplates GA formed from mixtures of d- and 1-amino acids.

In certain aspects, the present invention provides compositions comprising MDSCs and small compounds. For example, compositions comprising MDSCs in combination with GA and/or a MAP kinase inhibitor are provided. In a preferred embodiment, MDSCs are administered with GA and a MAP kinase inhibitor. In some aspects, MDSCs are derived from bone marrow or HSCs in vitro. In another aspect, MDSCs are freshly isolated from a patient or donor, as described, supra. The MDSCs of the invention may be autologous or allogeneic. In yet other aspects of the invention, a subject or patient is administered a composition containing MDSCs and one or more small compounds of the invention. Administration may be achieved by any suitable method. In yet another aspect, a subject is administered MDSCs and one or more small compounds of the invention, each as a separate composition. For example, a subject may be administered one composition containing MDSCs and one or more compositions each containing one or more small compound, such as, e.g., GA and/or SP600125. Such compositions may be administered to at the same or different times via the same or different routes of administration.

There are several therapeutic embodiments that are useful for the education of a practitioner of the invention. In one embodiment of the invention, a patient is administered a composition containing at least one stem cell mobilizer, such as, but not limited to G-CSF, AMD 3100, CTCE-9908, FTY720, Flt3 ligand, SCF, S100A9, GM-CSF and M-CSF. These agents would increase the amount of MDSC into circulation

The patient is further administered one or more additional compositions containing one or more small compounds of the invention for enhancing the suppressive activity of MDSCs, such as GA and/or SP600125. In certain aspects of the invention, these compositions may be administered at the same or different times and at the same or different sites. In another aspect, stem cell mobilizing agents and small compounds of the invention may be administered as a single composition. The compositions of the invention can be formulated for administration in any convenient way for use in human or veterinary medicine. The MDSCs of the invention may be incorporated into liposomes, microemulsions, micelles, unilamellar or multilamellar vesicles, erythrocyte ghosts or spheroblasts. In one embodiment, the MDSCs, stem cell mobilizing agents and/or small compounds of the invention can be delivered in one or more vesicles, including as a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

For the practice of the invention, in yet another embodiment, MDSCs and small compounds of the invention can be delivered in a controlled release form. In some example decellularized placental tissue is utilized as a delivery mechanism. There are other means of deliver that may be utilized, for example, one or more small compounds (e.g., GA and/or SP600125) may be administered in a polymer matrix such as poly (lactide-co-glycolide) (PLGA), in a microsphere or liposome implanted subcutaneously, or by another mode of delivery (see, Cao et al., 1999, Biomaterials, February; 20(4):329-39). Another aspect of delivery includes the suspension of the compositions of the invention in an alginate hydrogel. Additionally the use of micropumps is also disclosed.

When we spec of “therapeutically effective” we are referring to to a dose or an amount refers to that quantity of a compound or pharmaceutical composition that is sufficient to result in a desired activity upon administration to a mammal in need thereof. As used herein, the term “therapeutically effective amount/dose” refers to the amount/dose of a pharmaceutical composition of the invention that is suitable for treating a patient or subject having an autoimmune disease. In certain embodiments of the invention the patient or subject may be a mammal. In certain embodiments, the mammal may be a human.

The present invention also provides pharmaceutical formulations or dosage forms for administration to mammals in need thereof. The subject invention also concerns the use of GA or a GA derivative and/or MAP kinase inhibitors, such as, e.g., SP600125, in the preparation of a pharmaceutical composition. In some embodiments, a pharmaceutical composition of the invention includes MDSCs and GA and/or a small compound inhibitor of a MAP kinase. In a specific embodiment, the inhibitor is a small compound inhibitor of JNK. In yet another embodiment, the pharmaceutical composition includes MDSCs, GA and a small compound MAP kinase inhibitor. The pharmaceutical compositions of the invention optionally include a pharmaceutically acceptable carrier or diluent.

The compositions and formulations of the present invention can be administered topically, parenterally, orally, by inhalation, as a suppository, or by other methods known in the art. The term “parenteral” includes injection (for example, intravenous, intraperitoneal, epidural, intrathecal, intramuscular, intraluminal, intratracheal or subcutaneous). The preferred routes of administration are intravenous (i.v.), intraperitoneal (i.p.) and subcutaneous (s.c.) injection. When MDSCs are administered separately from the small compounds of the invention, the preferred route of administration is i.v. However, MDSCs may also be administered subcutaneously or intraperitoneally. The preferred route of administration for GA and the stem cell mobilizers of the invention is subcutaneous administration. The preferred route of administration for SP600125 is i.p. injection. However, the stem cell mobilizers and small compounds of the invention may be administered in any convenient way, including for i.v., s.c., oral, or i.p. injection. Administration of the compositions of the invention may be once a day, twice a day, or more often, but frequency may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

During the practice of the invention, it will be appreciated that the amount of MDSCs and small compounds of the invention required for use in treatment will vary with the route of administration, the nature of the condition for which treatment is required, and the age, body weight and condition of the patient, and will be ultimately at the discretion of the attendant physician or veterinarian. These compositions will typically contain an effective amount of the compositions of the invention, alone or in combination with an effective amount of any other active material, e.g., those described above. Preliminary doses can be determined according to animal tests, and the scaling of dosages for human administration can be performed according to art-accepted practices.] Keeping the above description in mind, typical dosages of MDSCs for administration to humans range from about 5.times.10.sup.5 to about 5.times.10.sup.6 or higher, although lower or higher numbers of MDSCs are also possible. In embodiments in which autologous MDSCs are administered, an advantage of the present invention is that there is little to no toxicity, since the MDSCs are autologous. In a preferred embodiment, a patient may receive, for example, 5.times.10.sup.7-5.times.10.sup.10 MDSCs. Keeping the above description in mind, typical dosages of GA for administration to humans may range from about 50 .mu.g/kg (of body weight) to about 50 mg/kg per day. A preferred dose range is on the order of about 100 .mu.g/kg/day to about 10 mg/kg/day, more preferably a range of about 300 .mu.g/kg/day to about 1 mg/kg/day, and still more preferably from about 300 .mu.g/kg/day to about 700 .mu.g/kg/day. The length of treatment, i.e., number of days, will be readily determined by a physician treating the patient, however the number of days of treatment may range from 1 day to about 20 days. In a preferred embodiment, the dose of GA is administered at a frequency of about once every 7 days to about once every day. In a more preferred embodiment, the dose of GA is administered at a frequency of about once every day. Preferably, the number of days of treatment is from about 5 to about 15 days and most preferably from about 10 to about 12 days. In a specific embodiment, a patient may receive, for example, 500 .mu.g/kg/day subcutaneously (SC) for 12 days. In another embodiment of the invention, the dose of GA is administered at a frequency of about once every 30 days to about once every day. In a specific embodiment, GA is administered subcutaneously for 12 days. [See, Weber, M. S., et al., (2007) Nat. Med.; 13(8):935-943.] Keeping the above description in mind, typical dosages of SP600125 for administration to humans range from 50 .mu.g/kg (of body weight) to about 500 mg/kg per day. A preferred dose is about 50 mg/kg/day. Keeping the above description in mind, typical dosages of the stem cell mobilizer Flt3 ligand may range from about 10 .mu.g/kg to about 1000 .mu.g/kg. A preferred dose range is on the order of about 20 .mu.g/kg to about 300 .mu.g/kg. In certain embodiments, a patient may receive, for example, 20 .mu.g/kg of Flt3L per day subcutaneously for 14 days each month [see Disis, M L et al. (2002) Blood. 99: 2845-2850]. Preferably, the length of treatment is at least 5 days. Keeping the above description in mind, typical dosages of G-CSF may range from about 2 to about 12 mg/kg/day. The length of treatment may range from about 1 day to about 14 days. Preferably, the length of treatment is at least 5 days. When formulated in a pharmaceutical composition, the compositions of the present invention can be admixed with a pharmaceutically acceptable carrier or excipient. The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicles with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Alternatively, the carrier can be a solid dosage from carrier, including but not limited to one or more of a binder (for compressed pills), an encapsulating agent, a flavorant, and a colorant. Suitable pharmaceutical carriers are described in “Remington's Pharmaceutical Sciences” by E. W. Martin. The compositions of the present invention can be formulated into any form known in the art using procedures available to one of skill in the art. The compositions of the present invention may be mixed with other food forms and consumed in solid, semi-solid, suspension or emulsion form. In one embodiment, the composition is formulated into a capsule or tablet using techniques available to one of skill in the art. However, the present compositions may also be formulated in another convenient form, such as an injectable solution or suspension, a spray solution or suspension, a lotion, a gum, a lozenge, a food or snack item. Food, snack, gum or lozenge items can include any ingestible ingredient, including sweeteners, flavorings, oils, starches, proteins, fruits or fruit extracts, vegetables or vegetable extracts, grains, animal fats or proteins. Thus, the present compositions can be formulated into cereals, snack items such as chips, bars, gum drops, chewable candies or slowly dissolving lozenges. The compositions of the present invention can also be administered as dry powder or metered dose of solution by inhalation, or nose-drops and nasal sprays, using appropriate formulations and metered dosing units.

In a specific embodiment, a pharmaceutical composition of the invention comprises: MDSCs in combination with GA or SP600125 alone, or MDSCs in combination with GA and SP600125, and a pharmaceutically acceptable carrier or diluent for intravenous or subcutaneous administration.

In some embodiments antioxidants may be administered in conjunction. Additionally, in some embodiments cells may be administered together with inhibitors of NF-kappa B. Known inhibitors include: Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

In yet another specific embodiment of the invention, a pharmaceutical composition of the invention comprises GA and/or SP600125 and a pharmaceutically acceptable carrier or diluent, and another pharmaceutical composition comprises MDSCs or one or more stem cell mobilizers. In still another embodiment, MDSCs, GA and/or a MAP kinase inhibitor, such as SP600125, are each administered separately to a patient in need of treatment as separate pharmaceutical compositions. In yet another embodiment, at least one stem cell mobilizer, and GA and/or a MAP kinase inhibitor, such as SP600125, arc each administered separately to a patient in need of treatment as separate pharmaceutical compositions. Any of the pharmaceutical compositions of the invention may be administered separately or together, at the same or different sites, at the same or different times, and according to the same or different frequencies of administration. In certain aspects of the invention, MDSCs arc pre-treated in vitro with GA or a MAP kinase inhibitor alone or with GA and a MAP kinase inhibitor. In other embodiments cells are pretreated with histone deaceylase inhibitors such as decitabine. In other embodiments, toll like receptor agonists may be utilized. The pre-treated MDSCs may then be administered to a patient in need of treatment. The effective amounts of GA and MAP kinase inhibitor for in vitro treatment of MDSCs may be readily determined by the skilled artisan without undue experimentation. In a titration experiment, the amount of GA and/or MAP kinase inhibitor that is effective for maintaining the MDSC in an immature state in the presence of the activator lipopolysaccharide (LPS) and is not toxic to the MDSC may be determined and selected for use.

In some embodiments of the invention, umbilical cord blood derived cells are utilized to generate MDSC for administration intra-articularly in patients suffering from cartilage degeneration. In one embodiment, he myeloid-derived suppressor cells of the present invention may be amplified and differentiated by culturing the CD34.sup.+ cells in a cell culture medium including GM-CSF and SCF for 2 weeks to 7 weeks, more specifically, for 3 weeks to 6 weeks. The cell culture medium may be a safe medium for animal cell culture. Ideally cell cultures are made under conditions of Good Manufacturing Practices (GMP).

Examples of the cell culture medium may include Dulbecco's Modified Eagle's Medium (DMEM), Minimal Essential Medium (MEM), Basal Medium Eagle (BME), RPMI1640, F-10, F-12, a Minimal Essential Medium (aMEM), Glasgow's Minimal Essential Medium (GMEM), Iscove's Modified Dulbecco's Medium, and the like, but the present invention is not limited thereto. The GM-SCF and SCF may be added to a cell culture medium at a concentration ratio of 1:0.8 to 0.3. Preferably, the GM-CSF may be added to a cell culture medium at a concentration of 50 ng/mL to 200 ng/mL. The SCF may be added to a cell culture medium at a concentration of 10 ng/mL to 100 ng/mL. Within these ranges, the proliferation of CD34.sup.+ cells may be relatively increased. According to an embodiment, when CD34.sup.+ cells are cultured in G-CSF/SCF for 3 weeks, the number of the CD34.sup.+ cells may be multiplied approximately 600-fold, but in GM-CSF/SCF, the number of the CD34.sup.+ cells may be multiplied 1,000 to 3,000-fold. Culturing of the CD34.sup.+ cells for inducing differentiation into myeloid-derived suppressor cells may be maintained for 2 weeks to 7 weeks, more preferably, for 3 weeks to 6 weeks, but the present invention is not limited thereto. According to an embodiment, when cultured for 3 weeks to 6 weeks, differentiation into myeloid-derived suppressor cells having 30% to 95% CD11b.sup.+CD33.sup.+ expression may be induced. The differentiation of the CD34.sup.+cells into myeloid-derived suppressor cells may be carried out in a CO.sub.2 incubator under conditions of a 5% to 15% carbon dioxide airflow quantity at 35 to 37.degree. C., but the present invention is not particularly limited thereto. Under the conditions, differentiation-induced and proliferated myeloid-derived suppressor cells may be proliferated to a cell number of 1,000 to 3,000-fold of an initial number of CD34.sup.+ cells during culturing. In the present specification, the term “myeloid-derived suppressor cell (MDSC)” refers to an immature myeloid cell which is present in an immature state because of a granulocyte or the like not completely differentiated in tumors, autoimmune diseases, and infections, and it was reported that the number of MDSCs increases in patients with an acute inflammatory disease, trauma, septicemia, or a parasitic or mycotic infection, as well as in cancer patients. The function of MDSCs is to effectively suppress activated T cells. It is known that the mechanism by which MDSCs regulate T cells is that a nitric oxide synthase, reactive oxygen species (ROS), and arginase which are an enzyme suppress T cell activation by maximizing the metabolism of L-arginine which is an essential amino acid. The myeloid-derived suppressor cells of the present invention, which were induced to differentiate from the CD34.sup.+ cells isolated from cord blood, may be monocytic myeloid-derived suppressor cells expressing cellular phenotypes of Lin.sup.−, HLA-DR.sup.low, and CD11b.sup.+CD33.sup.+. The myeloid-derived suppressor cells may express PDL-1, CCR2, CCR5, CD62L, CXCR4, and ICAM-1 as cell surface markers. According to an embodiment of the present invention, when the CD34.sup.+ cells isolated from cord blood were cultured in GM-CSF and SCF for 6 weeks and the cell surface thereof was stained, 70% HLA-ABC, 30% or less HLA-DR, and at least 90% CD45 were expressed, and compared to MDSCs whose differentiation was induced in a combination of G-CSF/SCF, 10% expression of CD83 and CD80 were observed only in the MDSCs whose differentiation was induced in a GM-CSF/SCF combination according to the present invention. CD86 was expressed at about 40% in the MDSCs of a GM-CSF/SCF combination, which showed an aspect of low expression of co-stimulatory molecules. In addition, CD40 was expressed at 40%, and CD1d, CD3, and B220, which are lymphocyte markers, were expressed at less than 5%. PDL-1 which is known to suppress the proliferation or activation of T cells was expressed at about 30% only in cells cultured in the GM-CSF/SCF combination. CD13 is a transmembrane glycoprotein which is expressed in a myeloid precursor, myeloperoxidase (MPO) is a protein in azurophilic granules of myeloid cells, and both are proteins which are expressed in MDSCs. The expression of CD13 was significantly increased in MDSCs induced by a GM-CSF/SCF combination compared to MDSCs induced by a G-CSF/SCF combination. MPO was expressed at 90% or more in all of the MDSCs induced by two different combinations. In addition, MDSCs induced by the combination of GM-CSF/SCF increase the expression of an immune suppressor substance selected from the group consisting of arginase 1, indoleamine 2,3-dioxygenase (IDO), and inducible nitric oxide synthase (iNOS), compared to MDSCs induced by a combination of G-CSF/SCF and human peripheral blood-derived dendritic cells. MDSCs induced by the combination of GM-CSF/SCF significantly suppress the proliferation of allogeneic CD4 T cells and thereby strongly reduce the secretion of IFN-.gamma. by antigen-specific T cell immune responses. It was observed that MDSCs induced by the combination of GM-CSF/SCF showed a significant increase in the secretion of IL-10 when stimulated with CD40 antibodies, and large amounts of VEFG and TGF-.beta. were secreted without being affected by whether or not stimulated with the CD40 antibodies. When CD4 T cells are stimulated by MDSCs in vitro, it is known that the number of Treg cells expressing FoxP3 increases, and when CD4 T cells are stimulated by MDSCs induced by a combination of GM-CSF/SCF, FoxP3 expression is confirmed, but IL-17 which is an inflammatory cytokine is not secreted. In addition, MDSCs induced by the combination of GM-CSF/SCF alleviate the degree of graft-versus-host disease, increases the survival rate, increases the secretions of serum anti-inflammatory cytokines, IL-10, and TGF-0, increases the secretions of anti-inflammatory proteins, CRP, MIP-3.beta., MMP-9, RANTES (CCL5), and SDF-1a, and suppresses inflammatory responses by reducing the secretions of inflammatory cytokines, IL-17, and IFN-.gamma., in an animal model for graft-versus-host disease. Moreover, the number of Treg cells expressing FoxP3 is increased. Therefore, the present invention provides a myeloid-derived suppressor cell, which is differentiated from a cord blood-derived CD34.sup.+ cell and proliferated, expresses cellular phenotypes of Lin.sup.−, HLA-DR.sup.low, and CD11b.sup.+CD33.sup.+, and expresses PDL-1, CCR2, CCR5, CD62L, CXCR4, and ICAM-1 as cell surface markers. In addition, the present invention provides an immunosuppressive composition, including the myeloid-derived suppressor cell which is monocytic. The myeloid-derived suppressor cell of the present invention may be used to prevent or treat a rejection response in organ transplantation or hematopoietic stem cell transplantation; an autoimmune disease; or an allergic disease, which is caused by a hypersensitive immune response. The immunosuppressive composition according to the present invention may further include a pharmaceutically acceptable carrier. The pharmaceutically acceptable carrier includes a carrier and a vehicle commonly used in the field of medicine, and specifically includes an ion exchange resin, alumina, aluminum stearate, lecithin, a serum protein (e.g., human serum albumin), buffer substances (e.g., various phosphates, glycine, sorbic acid, potassium sorbate, and partial glyceride mixtures of saturated vegetable fatty acids), water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substrates, polyethylene glycol, sodium carboxymethyl cellulose, polyarylates, wax, polyethylene glycol, wool grease, or the like, but the present invention is not limited thereto. In addition to the above components, the composition of the present invention may further include a lubricant, a wetting agent, an emulsifying agent, a suspending agent, a preservative, or the like. In one aspect, the composition according to the invention may be prepared with an aqueous solution for non-oral administration, and preferably, Hank's solution, Ringer's solution, or a buffer solution such as physically buffered saline may be used. A water-soluble injection suspension may include a substrate capable of increasing the viscosity of a suspension such as sodium carboxymethyl cellulose, sorbitol, or dextran. The composition of the present invention may be systemically or locally administered and may be formulated into an appropriate dosage form according to known techniques for such administration. For example, for oral administration, the composition may be mixed with an inert diluent or an edible carrier, sealed in a hard or soft gelatin capsule, or formulated as a tablet. In the case of oral administration, the active compound may be mixed with an excipient and used in the form of ingestible tablets, buccal tablets, troches, capsules, elixirs, suspensions, syrups, wafers, or the like. Various dosage forms for injection, parenteral administration, and the like may be prepared based on techniques known in the art or commonly practiced methods. For administration of a dosage form, intravenous injection, subcutaneous injection, intramuscular injection, peritoneal injection, percutaneous administration, and the like may be used. The appropriate dosage of the composition of the present invention may be variously prescribed depending on such factors as formulation method, administration method, the age, weight, gender, and morbidity of a patient, food, administration time, administration route, excretion rate, and reaction sensitivity. For example, the composition of the present invention may be administered to adults at a dosage of 0.1 to 1,000 mg/kg, preferably at a dosage of 10 to 100 mg/kg, once or several times daily.

In some embodiments exosomes are isolated from MDSC, concentrated, and administered to induce regeneration of the cartilage. In one embodiment, MDSC are cultured using means known in the art for preserving viability and proliferative ability of MDSC. The disclosed methods may be applied both for individualized autologous exosome preparations and for exosome preparations obtained from established cell lines, for experimental or biological use. In one embodiment, methods of the disclosure encompass the use of chromatography separation methods for preparing membrane vesicles, particularly to separate the membrane vesicles from potential biological contaminants, wherein the microvesicles are exosomes, and cells utilized for generating said exosomes are fibroblast cells. The exosomes are obtained and may be prepared for administration to one or more individuals in need thereof. Indeed, the applicant has now demonstrated that membrane vesicles, particularly exosomes, could be purified, and possess ability to inhibit pain. In one embodiment, a strong or weak, preferably strong, anion exchange may be performed. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE POROS™ SEPHAROSE™, SEPHADEX™, TRISACRYL™, TSK-GEL SW OR PW™, SUPERDEX™ TOYOPEARL HW and SEPHACRYL™, for example, which are suitable for the application of this invention. Therefore, in a specific embodiment, this invention relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the disclosure, one can use supports in bead form. In particular embodiments, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, one can use high porosity gels, particularly between 10 nm and 5 .mu.m, such as between approximately 20 nm and approximately 2 .mu.m, including between about 100 nm and about 1 .mu.m. For the anion exchange chromatography, the support used may be functionalized using a group capable of interacting with an anionic molecule. Generally, this group comprises an amine that may be ternary or quaternary, which defines a weak or strong anion exchanger, respectively. Within the scope of this disclosure, one can utilize a strong anion exchanger. In this way, according to the disclosure, a chromatography support as described above, functionalized with quaternary amines, may be used. Therefore, according to a more specific embodiment of the disclosure, the anion exchange chromatography is performed on a support functionalized with a quaternary amine. In specific cases, this support is selected from poly(styrene-divinylbenzene), acrylamide, agarose, dextran and silica, alone or in mixtures, and may be functionalized with a quaternary amine. Examples of supports functionalized with a quaternary amine include the gels SOURCEQ. MONO Q, Q SEPHAROSE™, POROS™ HQ and POROSR™ QE, FRACTOGEL™. TMAE type gels and TOYOPEARL SUPER™ Q gels. One example of a support to perform the anion exchange chromatography comprises poly(styrene-divinylbenzene). An example of this type of gel that may be used within the scope of this disclosure is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way may be detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 .mu.1 up to 10 ml or greater. In this way, the supports available have a capacity which may reach 25 mg of proteins/ml, for example. For this reason, a 100 .mu.1 column has a capacity of approximately 2.5 mg of proteins which, given the samples in question, allows the treatment of culture supernatants of approximately 2 1 (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the disclosure, a gel permeation chromatography step is added to the anion exchange step, either before or after the anion exchange chromatography step. In this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, may be used. As an illustration, for gel permeation chromatography, a support such as SUPERDEX™ 200HR (Pharmacia), TSK G6000 (TosoHaas) or SEPHACRYL™ S (Pharmacia) may be used. The process according to the disclosure may be applied to different biological samples. In particular, these may comprise a biological fluid from a subject (bone marrow, peripheral blood, etc.), a culture supernatant, a cell lysate, a pre-purified solution or any other composition comprising membrane vesicles.

In this respect, in a specific embodiment of the disclosure, the biological sample is a culture supernatant of membrane vesicle-producing MDSC. In addition, according to one embodiment of the disclosure, the biological sample is treated, prior to the chromatography step, to be enriched with membrane vesicles (enrichment stage). In this way, in a specific embodiment, this disclosure relates to a method of preparing membrane vesicles from a biological sample, characterized in that it comprises at least: a) an enrichment step, to prepare a sample enriched with membrane vesicles, and b) a step during which the sample is treated by anion exchange chromatography and/or gel permeation chromatography. In one embodiment, the biological sample is a culture supernatant treated so as to be enriched with membrane vesicles. In particular, the biological sample may be comprised of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, one method of preparing membrane vesicles according to this disclosure more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In one specific embodiment, the enrichment step comprises an affinity chromatography step, optionally preceded by a step of elimination of cells and/or cell debris (clarification). An example of an enrichment step according to this disclosure comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 .mu.m, e.g. between 0.2 and 10 .mu.m, may be used. It is particularly possible to use a succession of filters with a porosity of 10 .mu.m, 1 .mu.m, 0.5 .mu.m followed by 0.22 .mu.m.

A concentration step may also be performed, such as in order to reduce the volumes of sample to be treated during the chromatography stages. In this way, the concentration may be obtained by centrifugation of the sample at high speeds, e.g., between 10,000 and 100,000 g, to cause the sedimentation of the membrane vesicles. This may comprise a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a particular embodiment, the biological sample (e.g., the supernatant) is subjected to an ultrafiltration, preferably a tangential ultrafiltration. Tangential ultrafiltration comprises concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. In some cases, an affinity chromatography on a dye is used, allowing the elimination (i.e., the retention) of contaminants such as proteins and enzymes, for instance albumin, kinases, dehydrogenases, clotting factors, interferons, lipoproteins, or also co-factors, etc. More preferably, the support used for this chromatography step is a support as used for the ion exchange chromatography, functionalized with a dye. As specific example, the dye may be selected from Blue SEPHAROSE™ (Pharmacia), YELLOW 86, GREEN 5 and BROWN 10 (Sigma). The support may be agarose. It should be understood that any other support and/or dye or reactive group allowing the retention (binding) of contaminants from the treated biological sample can be used in the instant disclosure.

In one embodiment a membrane vesicle preparation process within the scope of this disclosure comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a certain embodiment, step b) above comprises a filtration of the culture supernatant, followed by an ultrafiltration, such as tangential. In another embodiment, step b) above comprises a clarification of the culture supernatant, followed by an affinity chromatography on dye, such as on Blue SEPHAROSE™.

In addition, after step c), the material harvested may, if applicable, be subjected to one or more additional treatment and/or filtration stages d), particularly for sterilization purposes. For this filtration treatment stage, filters with a diameter less than or equal to 0.3 .mu.m may be used, or for example, less than or equal to 0.25 .mu.m. Such filters have a diameter of 0.22 .mu.m, for example. After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the disclosure comprises at least the following steps: c) an anion exchange chromatography and/or gel permeation chromatography treatment of the biological sample, and d) a filtration step, particularly sterilizing filtration, of the material harvested after stage c). In a first variant, the process according to the disclosure comprises: c) an anion exchange chromatography treatment of the biological sample, and d) a filtration step, particularly sterilizing filtration, on the material harvested after step c). In some cases, instead of being stored the material may be used for one or more individuals in the absence of a prior storage step.

In another variant, the process according to the disclosure comprises: c) a gel permeation chromatography treatment of the biological sample, and d) a filtration step, particularly sterilizing filtration, on the material harvested after step c). According to a third variant, the process according to the disclosure comprises: c) an anionic exchange treatment of the biological sample followed or preceded by gel permeation chromatography, and d) a filtration step, particularly sterilizing filtration, on the material harvested after step c). Further embodiments include a method of optimizing one or more pain inhibiting therapeutic factors production from fibroblast cultures through the use of filters that separate compositions based on electrical charge, size and/or ability to elute from an adsorbent. Numerous techniques are known in the art for purification of therapeutic factors and concentration of agents. For some particular uses fibroblast derived compounds are sufficient for use as culture supernatants of the cells in media. Currently media useful for this purpose include Roswell Park Memorial Institute (RPMI-1640) Dublecco's Modified Essential Media (DMEM), Eagle's Modified Essential Media (EMEM), Optimem, and Iscove's Media. In one embodiment, therapeutic factors capable of inhibiting disc degeneration or cartilage degeneration, are derived from tissue culture that may comprise exosomes, or may not comprise exosomes but comprise one or more factors capable of stimulating a regenerative effect. In such an embodiment, culture conditioned media may be concentrated by filtering/desalting means known in the art including use of Amicon filters with specific molecular weight cut-offs, said cut-offs may select for molecular weights higher than 1 kDa to 50 kDa. Supernatant may alternatively be concentrated using means known in the art such as solid phase extraction using C18 cartridges (Mini-Spe-ed C18-14%, S.P.E. Limited, Concord ON). The cartridges may be prepared by washing with methanol followed by deionized-distilled water. Up to 100 ml of fibroblast conditioned media supernatant may be passed through each of these specific cartridges before elution, and it is understood of one of skill in the art that larger cartridges may be used. After washing the cartridges, material adsorbed is eluted, such as with 3 ml methanol, evaporated under a stream of nitrogen, re-dissolved in a small volume of methanol, and stored at 4.degree. C. Before testing the eluate for activity in vitro, the methanol is evaporated under nitrogen and replaced by culture medium. The C18 cartridges may be used to adsorb small hydrophobic molecules from the fibroblast conditioned supernatant and allows for the elimination of salts and other polar contaminants. It may, however be desired to use other adsorption means in order to purify certain compounds from the supernatant. The concentrated supernatant may be assessed directly for biological activities useful for the practice of this disclosure, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Fibroblast cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

1. A method of treatment cartilage degeneration comprising administration of myeloid derived suppressor cells and/or exosomes generated under stimulated or unstimulated conditions from said myeloid derived suppressor cells.

2. The method of claim 1, wherein myeloid derived suppressor cells express Grp1.

3. The method of claim 1, wherein myeloid derived suppressor cells express interleukin-3 receptor.

4. The method of claim 1, wherein myeloid derived suppressor cells are capable of inhibiting T cell proliferation.

5. The method of claim 4, wherein T cell proliferation is stimulated by interleukin-18.

6. The method of claim 4, wherein said T cell proliferation is stimulated by anti-CD3 antibody.

7. The method of claim 4, wherein said T cell proliferation is stimulated by anti-CD28 antibody.

8. The method of claim 1, wherein said myeloid derived suppressor cells are progenitors of granulocytes.

9. The method of claim 1, wherein said myeloid derived suppressor cells are progenitors of monocytes.

10. The method of claim 1, wherein said myeloid derived suppressor cells are progenitors of dendritic cells.

11. The method of claim 1, wherein said myeloid derived suppressor cells are collected from apheresed product after mobilization.

12. The method of claim 11, wherein said mobilization is achieved by administration of SCF.

13. The method of claim 1, wherein said exosomes are derived from said cell population is autologous.

14. The method of claim 13, wherein said cell population is allogeneic.

15. The method of claim 13, wherein said cell population is bone marrow aspirate/mononuclear cells.

16. The method of claim 13, wherein said cell population is mesenchymal stem cells.

17. The method of claim 13, wherein said cell population is inducible pluripotent stem cells.

18. The method of claim 16, wherein said mesenchymal stem cells express IL-1 receptor.

19. The method of claim 16, wherein said mesenchymal stem cells express IL-1 receptor antagonist when treated with interferon gamma.

20. The method of clam 1, wherein said cell population is administered before or after intraarticular laser treatment.
